(19) United States
(12) Patent Application Publication
(10) Pub. No.: US 2004/0020796 A1
(43) Pub. Date: Feb. 5, 2004

(54) Dental material container

(76) Inventors: Jeffery James Cheetham, East Malvern (AU); Joshua James Cheetham, Prahran East (AU); Craig Wesley Hoenderop, Lilydale (AU)

Correspondence Address: LAW OFFICES OF WILLIAM H. HOLT, Unit 2, First Floor, 1423 Powhatan Street, Alexandria, VA, 22314, US

(21) Appl. No.: 10/629,573

(22) Filed: Jul. 30, 2003

(30) Foreign Application Priority Data
Aug. 1, 2002 (AU) 2002950515
Mar. 13, 2003 (AU) 2003901135

(51) Int. Cl. A61B 19/02

(52) U.S. Cl. 206/63.5; 206/219

(57) ABSTRACT

A dental material container comprising a body including a first chamber and a second chamber separated by a first wall member. A dispensing member is provided separated from the second chamber by a second wall member. Also provided is a means for applying pressure to dental material contained within the first chamber. Actuation of the means for applying pressure causes dental material within the first chamber to be expelled through the first wall member and mix with dental material in the second chamber. Further actuation of the means for applying pressure causes the mixed dental material to be expelled through the second wall member to the dispensing member.

The present invention relates to a dental material container.

In accordance with one aspect of the present invention there is provided a dental material container comprising a body including a first chamber and a second chamber separated by a first wall member, a dispensing member separated from the second chamber by a second wall member, and a means for applying pressure to dental material contained within the first chamber, wherein actuation of the means for applying pressure causes dental material within the first chamber to be expelled through the first wall member and mix with dental material in the second chamber and further actuation of the means for applying pressure causes the mixed dental material to be expelled through the second wall member to the dispensing member.

The present invention will now be described, by way of example, with reference to the accompanying drawings, in which:

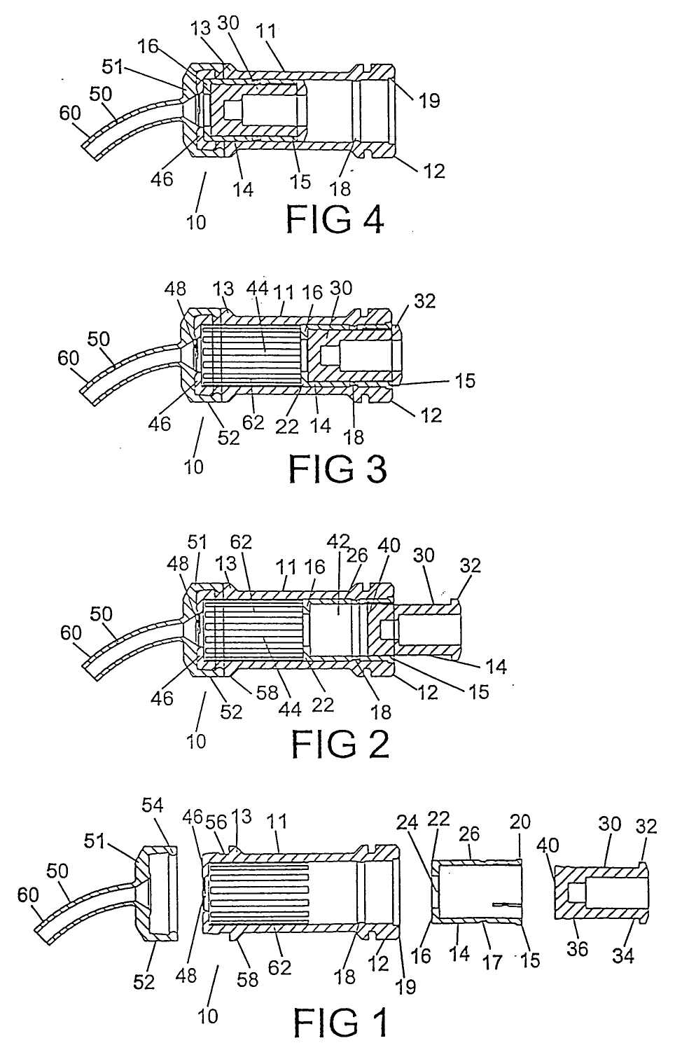

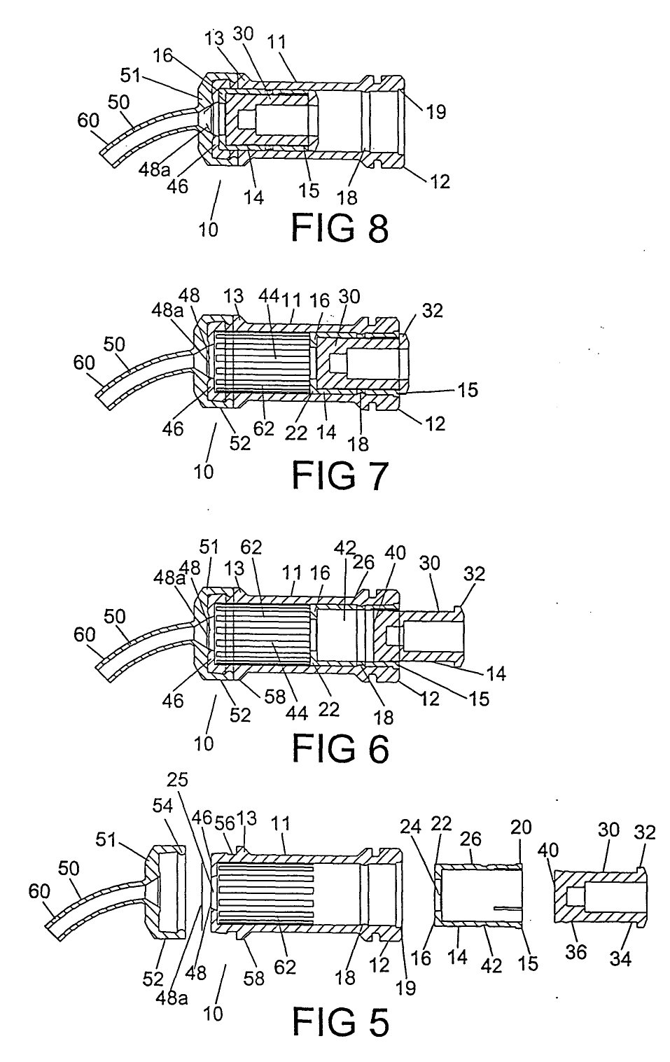

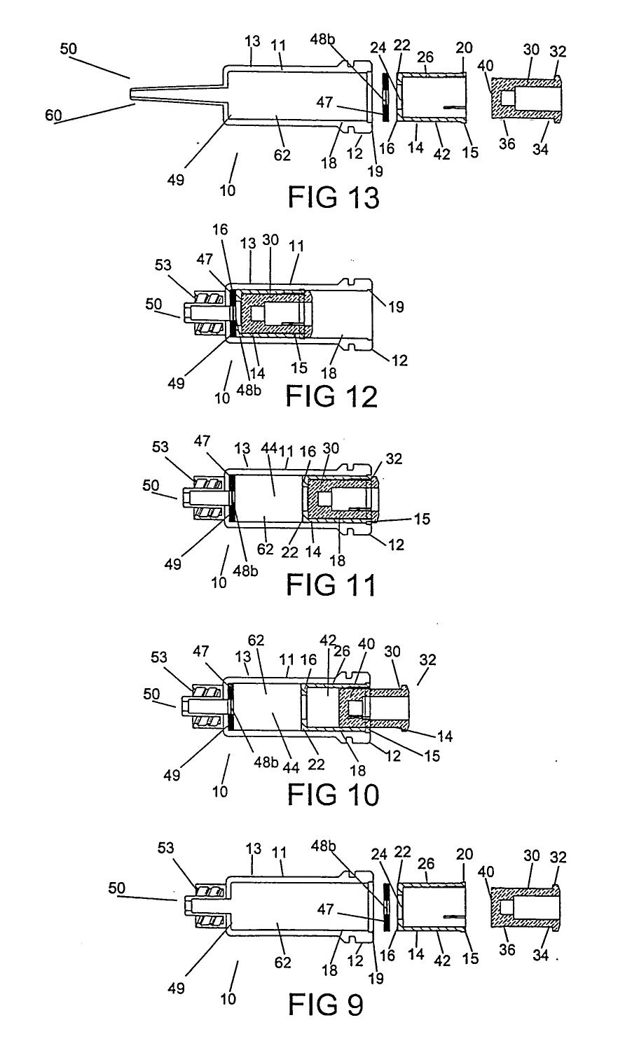

FIG. 1 is a side view of a container in accordance with the present invention in section and in disassembled condition;

FIG. 2 is a view similar to FIG. 1 with the components of the container in assembled condition;

FIG. 3 is a view similar to FIG. 2 in which the container is partially activated;

FIG. 4 is a view similar to FIG. 3 in which the container is fully activated;

FIG. 5 is a side view of an alternative embodiment of a container in accordance with the present invention in section and in disassembled condition;

FIG. 6 is a view similar to FIG. 5 with the components of the container in assembled condition;

FIG. 7 is a view similar to FIG. 6 in which the container is partially activated;

FIG. 8 is a view similar to FIG. 7 in which the container is fully activated;

FIG. 9 is a side view of an further embodiment of a container in accordance with the present invention in section and in disassembled condition;

FIG. 10 is a view similar to FIG. 9 with the components of the container in assembled condition;

FIG. 11 is a view similar to FIG. 10 in which the container is partially activated;

FIG. 12 is a view similar to FIG. 11 in which the container is fully activated; and

FIG. 13 is a view similar to FIG. 12 in which the container includes an integral dispensing means.

In the drawings, there is shown a dental material container 10 which has a hollow generally cylindrical body 11. The body 11 has a first end 12 and second end 13. A receptacle 14 is arranged to be inserted into the body 11 at the first end 12 thereof as shown in FIG. 2. The receptacle 14 is of less length than the body 11 and has a first open end 15 and a second end 16. Further the receptacle 14 is hollow and of generally cylindrical shape. The receptacle 14 has a cylindrical wall section 26 which is internally smooth. Also, the wall section 26 of the receptacle 14 is provided with a circumferential groove 17 which engages, in use, with a corresponding internal circumferential rib 18 in the body 11.

Further, the body 11 at the first end 12 thereof is provided with a circumferential recess 19. The recess 19 engages in use with an outwardly extending circumferential rib 20 at the first end 15 of the receptacle 14. Thus, as shown in FIGS. 2, 6 and 10 when assembled the receptacle 14 extends partially into the body 11 with the groove 17 in engagement with the rib 18. At the second end 16 of the receptacle 14 there is provided a transverse first wall member 22 which has a central weakened portion 24. The weakened portion 24 may be formed of a relatively thin portion of the first wall member 22. The second end 16 of the receptacle 14 is, as shown, inserted foremost into the body 11 at the first end 13 thereof.

Further, a plunger 30 is arranged to be inserted into the receptacle 14 when the latter is assembled as shown in FIGS. 2, 6 and 10. The plunger 30 is of generally cylindrical external shape and has an outwardly extending flange 32 at an outer end 34. Initially, as shown in FIG. 2, the plunger 30 is inserted partially into the receptacle 14.

Still further, an inner end 40 of the plunger 30 is spaced from the first wall member 22 in the initial position of the plunger 30 to form a first chamber 42. A second chamber 44 is located between the first wall member 22 and the second end 13 of the body 11. The second end 13 of the body 11 is formed with an end second wall member 46 having a central weakened portion 48. The second end wall 46 may be provided as a wall extending across the body 11 to close off the end 13 thereof with a weakened portion 48 being formed by a portion of relatively thin material compared to the remainder of the second wall member 46 as shown in FIGS. 1 to 4. Alternatively, the weakened portion may be provided by the second end 13 of the body having an opening 25 across which is secured a membrane 48a, as shown in FIGS. 5 to 8.

Further, a dispensing member 50 is mounted to the second end 13 of the body 11. The dispensing member 50 of FIGS. 1 to 8 has a base 51 provided with a recess surrounded by a cylindrical wall 52. The cylindrical wall 52 fits over the second end 13. Also, the cylindrical wall 52 has an internal circumferential rib 54 which snap fits with a circumferential groove 56 (see FIG. 1) on the body 11 adjacent the second end 13. Further, the body 11 is provided with a circumferential flange 58 against which the wall cylindrical 52 abuts in the assembled condition shown in FIGS. 2, 6 and 10.

Further, the dispensing member 50 is provided with a curved dispensing nozzle 60 which may be of substantially uniform internal thickness. Alternatively, the dispensing nozzle 60 may be tapered so as to have a smaller end internal thickness.

In a further embodiment of the invention, as shown FIGS. 9 to 12, the body 11 is provided with an integral dispensing member 50. In this embodiment, the dispensing member 50 is provided with a screw connection 53 to which can be secured a dispensing nozzle 60 (not shown). The dispensing nozzle 60 may comprise a bent tube attached to a plastic hub arranged to connect to the screw connection 53. In this embodiment, the second wall 46 is provided as a separate disc member 47 which is arranged to be inserted into the body 11 where is rests against an internal shoulder 49 of the body 11 adjacent the dispensing member 50. The disc member 49 is provided with a central weakened portion 48b.

FIG. 13 shows a container similar to that of FIG. 9 wherein the container includes an integral dispensing nozzle 60. The integral dispensing nozzle 60 may also be curved or angled with respect to the body.

In use, the container 10 is assembled in the condition shown in FIG. 2. Further, a quantity of particulate material is placed in the container 10 before the receptacle 14 is engaged with the body 11. Thus, the particulate material is contained in the second chamber 44 in the condition of the container shown in FIGS. 2, 6 and 10. Then a quantity of liquid is placed in the receptacle 14 before the plunger 30 is engaged therewith. Thus, the first chamber 42 contains the quantity of liquid in the condition shown in FIGS. 2, 6 and 10.

Subsequently, when the container 10 is used the plunger 30 is manually depressed until it reaches the position shown in FIGS. 3, 7 and 11 at which the end 40 abuts the first wall member 22. This action applies pressure to the quantity of liquid which correspondingly applies hydraulic pressure to the first wall member 22. This causes the weakened portion 24 to rupture and the quantity of liquid to be expelled into the second chamber 44. The liquid is thus admixed with the particulate material. The liquid and particulate material are thoroughly admixed into a homogenous dental composition by placing the container 10 in the condition shown in FIG. 3 into a vibratory mixer of known type.

Then the container 10 is removed from the mixer and the plunger 30 is depressed further to the position shown in FIGS. 4, 8 and 12 at which the end 40 abuts the second wall member 46. This action causes the dental composition to apply pressure to the weakened portion 48 of the second end wall 46. The weakened portion 48 is ruptured and the dental composition is expelled through the dispensing nozzle 60 of the dispensing member 50 directly into a tooth of a patient.

The container of the present invention is particularly envisaged for use with dental materials.

Further, as shown in the drawings the body 11 may be formed with a plurality of internal longitudinally extending grooves 62 in the chamber 44 to assist in expelling air from the chamber 44 upon movement of the plunger 30 to the position shown in FIG. 4. While the grooves 62 shown in the Figures extend only part way along the body 11, the grooves 62 may extend all the way to the first end 12. Other known means of expelling air from the body 11 such as uneven or roughly textured internal surfaces may also be employed.

Modifications and variations as would be apparent to a skilled addressee are deemed to be within the scope of the present invention

The claims defining the invention are as follows:

1. A dental material container comprising a body including a first chamber and a second chamber separated by a first wall member, a dispensing member separated from the second chamber by a second wall member, and a means for applying pressure to dental material contained within the first chamber, wherein actuation of the means for applying pressure causes dental material within the first chamber to be expelled through the first wall member and mix with dental material in the second chamber and further actuation of the means for applying pressure causes the mixed dental material to be expelled through the second wall member to the dispensing member.

2. A dental material container in accordance with claim 1, wherein the body has a first open end and a second end adjacent which is located the dispensing means, the first and second chambers being located within the body in use and the means for applying pressure comprising a plunger arranged to be inserted into the first open end of the body.

3. A dental material container in accordance with claim 2, wherein the second wall member is provided adjacent the second end of the body and the dental material container includes a receptacle arranged to be inserted into the open end of the body, the receptacle including the first wall member and having an open end arranged to receive the plunger such that the first chamber is defined between the plunger and the first wall member and the second chamber is defined between the first wall member and the second wall member.

4. A dental material container in accordance with claim 3, wherein the body of the dental material container and the receptacle are cylindrical and the first wall member is arranged transversely of the longitudinal axis of the cylindrical receptacle at an opposite end from the open end of the receptacle.

5. A dental material container in accordance with claim 4, wherein the receptacle includes a circumferential rib adjacent the open end thereof arranged to engage with a circumferential recess adjacent the first end of the body when the receptacle is inserted into the body.

6. A dental material container in accordance with claim 1, wherein the first wall member includes a central weakened portion.

7. A dental material container in accordance with claim 6, wherein the central weakened portion comprises a relatively thin portion of the first wall member.

8. A dental material container in accordance with claim 1, wherein the second wall member includes a central weakened portion.

9. A dental material container in accordance with claim 8, wherein the central weakened portion comprises a relatively thin portion of the second wall member.

10. A dental material container in accordance with claim 1, wherein the dispensing member includes a dispensing nozzle and a circumferential rib arranged to snap fit with a circumferential groove adjacent the body of the dental material container.

11. A dental material container in accordance with claim 1, wherein the dispensing member is integrally formed with the body of the dental material container and includes a dispensing nozzle.

12. A dental material container in accordance with claim 1, wherein the dispensing member includes a screw connection on the body of the dental material container and a dispensing nozzle arranged to engage with the screw connection.

13. A dental material container in accordance with claim 1, wherein the dispensing nozzle is curved.

14. A dental material container in accordance with claim 1, wherein the second wall member is provided as a separate disc member arranged to be inserted into the body of the dental material container.

15. A dental material container in accordance with claim 1, wherein the body of the dental material container includes a means for expelling air from within the body during use.

16. A dental material container in accordance with claim 15, wherein the means for expelling air comprises longitudinal grooves on inner surfaces of the body.
